(19) United States
(12) Patent Application Publication
(10) Pub. No.: US 2021/0030397 A1
(43) Pub. Date: Feb. 4, 2021

(54) Medical Device with Structured Echogenic Coating Application and Method for Preparing Same

(71) Applicant: Avent, Inc., Alpharetta, GA (US)

(72) Inventors: Sung Lee, Irvine, CA (US); Curry B. Sandven, Cumming, GA (US)

(21) Appl. No.: 16/527,641

(22) Filed: Jul. 31, 2019

(51) Int. Cl. A61B 8/00; A61B 8/08; A61K 49/22; A61L 27/56

(52) U.S. Cl. CPC A61B 8/4455; A61B 8/08; A61B 2090/3925; A61L 27/56; A61K 49/22

(57) ABSTRACT

An echogenically enhanced medical device is provided. The echogenically enhanced medical device includes a body portion configured to be inserted within human or animal tissue, where the body portion has a first acoustic impedance. The body portion of the medical device further includes at least one structured echogenic portion having a second acoustic impedance. The at least one structured echogenic portion is configured to enhance ultrasonic imaging of the medical device when inserted into a patient. The at least one structured echogenic portion includes at least one of a structured shape and a discontinuous pattern. A method of preparing an echogenically enhanced medical device is further disclosed.

### FIELD OF THE INVENTION

The subject matter of the present invention relates generally to an echogenically enhanced medical device and method for preparing an echogenically enhanced medical device.

### BACKGROUND

Ultrasonic imaging in the medical field is widely used for a variety of applications. In addition to imaging physiological structures and tissue such as organs, tumors, vessels, and the like, it is often desirable for a physician or technician to view an image of a medical device which has been inserted into the tissue or passageway of a patient. The types of devices which are surgically sterilized and inserted into patients are many. Typical examples include: needles, catheters, and a variety of other medical products such as stents, dilators, pacing leads, introducers, angiography devices, angioplasty devices, pacemakers, in-patient appliances such as pumps and other devices. For example, during a nerve block procedure, visibility of such medical devices under ultrasound is particularly important. More specifically, in such procedures, locating the catheter tip can be critical around the nerve site. Various approaches have been used to enhance ultrasonic imaging of such devices by modifying the reflective surface characteristics of these devices, such as by altering the geometry of the surface, increasing surface roughness, and fabricating surfaces which may entrap gas.

For example, one existing solution is to provide an echogenic coating on a medical device. A portion of the medical device is covered with a polymeric coating, e.g., a polymeric coating formed from a porous material or microporous material. The echogenic coating has small bubbles, voids, spheres or other porous structures of a non-metallic material. However, the application of such echogenic coatings over a surface of the medical device are limited by the process of dipping or spray coating the echogenic material, which can only adapt the coating to a conformal shape of a catheter or cannula. For instance, when a needle or catheter is coated with an echogenic material, the shape of the coated area can resemble a screw or a bone shape when ultrasonic imaging is used during a medical procedure, such as a nerve block. This can result in a clinician inserting a catheter for a nerve block procedure in an inaccurate location. Moreover, using masking methods, e.g., stenciling, to create a pattern in the coating is limited to very simple shapes such as a band or a rectangle due to the typically very small size of the medical device, e.g., catheter tip or needle, relative to the size of the masking that is able to be achieved. Smaller and/or more precise or intricate patterns and shapes are impossible to create even with existing masking methods. Unfortunately, such band or rectangular shapes formed using masking methods may still result in an inconclusive ultrasound image in which the medical device appears confusingly similar to a bone or titanium screw.

Consequently, there is a need for echogenically enhancing a medical device in a manner that does not result in any potential confusion with other articles within the body, such as bones or screws. In particular, echogenically enhancing a medical device with a distinct shape and/or pattern in order to positively identify the medical device that is being positioned would also be useful.

### SUMMARY

The present invention is directed to an echogenically enhanced medical device. The echogenically enhanced medical device includes a body portion configured to be inserted within human or animal tissue, the body portion having a first acoustic impedance. The body portion further includes at least one structured echogenic portion having a second acoustic impedance configured to enhance ultrasonic imaging of the medical device when inserted into a patient. The at least one structured echogenic portion includes at least one of a structured shape and a discontinuous pattern.

In one particular embodiment, the body portion includes one of: a needle; a catheter; a cannula; a guidewire; an electrical lead; an implantable device; a probe; an implantable fluid delivery port; a breathing tube.

In another embodiment, the body portion is formed from one or more of metal material, plastic material, ceramic material, and resin-based material.

In a further embodiment, the second acoustic impedance is different from the first acoustic impedance.

In yet another embodiment, the at least one echogenic portion is formed from a material that is different from a material of the body portion.

In an additional embodiment, the discontinuous pattern of the echogenic portion is formed by additive or subtractive methods. Further, the structured shape or discontinuous pattern can be formed by printing an echogenic material onto the body portion. Moreover, the structured shape or discontinuous pattern can be imprinted by using a laser to remove material in the echogenic portion.

In still another embodiment, the at least one structured shape or discontinuous pattern is a symbol in the form of an arrow, an alphabetical letter, a number, a serial number, a date, a bullseye, a QR code, a bar code, a logo, or other shape configured to indicate the identity of the medical device.

In a further embodiment, the at least one structured shape or discontinuous pattern is a combination of alphabetical letters and/or numbers forming a date.

In an additional embodiment, the structured echogenic portion is formed by coating a portion of the body portion with an echogenic material.

In one more embodiment, the echogenic portion is a portion of the body portion formed from an echogenic material.

The present invention is further directed to a method for preparing an echogenically enhanced medical device. The method includes steps of: providing a medical device having a body portion configured to be inserted within human or animal tissue, the body portion having a first acoustic impedance; forming a structured echogenic portion having a second acoustic impedance, wherein the structured echogenic portion is configured to enhance ultrasonic imaging of the medical device when inserted into a patient, wherein the at least one structured echogenic portion comprises at least one of a shape and a discontinuous pattern.

In one particular embodiment, the step of forming a structured echogenic portion includes applying a coating of an echogenic material onto at least a portion of the body portion of the medical device. Further, the method can include a step of subtractively removing echogenic material from the echogenic coating to form the at least one of a shape and a discontinuous pattern. Moreover, the method can include a step of printing at least one additive structure onto the coating of echogenic material to form at least one of a shape and a discontinuous pattern.

In another embodiment, the at least one structured shape or discontinuous pattern is a symbol in the form of an arrow, an alphabetical letter, a number, a serial number, a date, a bullseye, a QR code, a barcode, a logo, or other shape configured to indicate the identity of the medical device.

In an additional embodiment, the at least one structured shape or discontinuous pattern is a combination of alphabetical letters and/or numbers forming a date.

In a further embodiment, the at least one of a shape and a discontinuous pattern is additively formed on the body portion of the medical device. Further, at least one additive structure can be printed on the body portion of the medical device to form the at least one of a shape and a discontinuous pattern.

The present invention is further directed to a method for identifying an echogenically enhanced medical device within a patient's body. The method includes the steps of: providing a medical device having a body portion configured to be inserted within human or animal tissue, the body portion having a first acoustic impedance; forming at least one structured echogenic portion on the medical device body portion, the structured echogenic portion having a second acoustic impedance, wherein the structured echogenic portion is configured to enhance ultrasonic imaging of the medical device when inserted into a patient, wherein the at least one structured echogenic portion comprises at least one of a shape and a discontinuous pattern; inserting the body portion of the medical device into the patient's body; receiving data signals from an autonomous ultrasound imaging system, the data signals comprising information related to a plurality of ultrasound waves generated by an ultrasound probe of the autonomous ultrasound generating system; and identifying the structured echogenic portion of the body portion of the medical device using the received data signals by identifying the at least one of a shape and a discontinuous pattern of the structured echogenic portion.

These and other features, aspects, and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 illustrates a perspective view of an echogenically enhanced medical device according to one particular embodiment of the present invention;

FIG. 2 illustrates a perspective view of another embodiment of an echogenically enhanced medical device according to the present invention;

FIG. 3 illustrates a cross-sectional view of an echogenically enhanced medical device of the present invention having an additive structured echogenic coating;

FIG. 4 illustrates a cross-sectional view of an echogenically enhanced medical device of the present invention having a subtractive structured echogenic coating;

FIG. 5 illustrates a perspective view of yet another embodiment of an echogenically enhanced medical device according to the present invention; and

FIG. 6 illustrates a block diagram of a method for preparing an echogenically enhanced medical device and identifying the echogenically enhanced medical device within a patient's body.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

The positional terms “proximal” and “distal” are used herein to orient the various components relative to each other and to the patient. “Distal” refers to the direction that is closest to the wound site and/or the patient (e.g., the distal end of the catheter or needle is the end oriented towards a catheter or needle insertion site of the patient), and “proximal” refers to the opposite direction (e.g., the proximal end of the catheter is inserted into the distal end of a fluid delivery device).

As used herein, “echogenic” means giving rise to reflections or echoes of ultrasound waves. “Echogenicity” refers to the relative extent that a surface reflects ultrasound energy directly back to a sensor, which is proximal to the source or emitter of the ultrasonic energy. The low practical echogenicity of a device, e.g., a medical device, hampers accurate imaging of the device within a medium. When the smooth device is oriented at right angles to ultrasound waves, the ultrasound waves are directly reflected off the device back to the ultrasound transducer, and the device is said to have a relatively high practical echogenicity. At other orientation angles, less of the ultrasound energy is directly reflected back to the transducer, thus reducing the practical echogenicity of the device.

As used herein, the terms “about,” “approximately,” or “generally,” when used to modify a value, indicates that the value can be raised or lowered by 5% and remain within the disclosed embodiment.

Generally speaking, the present invention is directed to an echogenically enhanced medical device. The echogenically enhanced medical device includes a body portion configured to be inserted within human or animal tissue, where the body portion has a first acoustic impedance. The body portion further includes at least one echogenic portion having a second acoustic impedance that is configured to enhance ultrasonic imaging of the medical device when inserted into a patient. The at least one echogenic portion includes at least one of a shape and a discontinuous pattern formed from an echogenic material. The present invention is further directed to methods of preparing an echogenically enhanced medical device having at least one of a shape and a discontinuous pattern formed from an echogenic material. The present inventors have found that the specific components of the echogenically enhanced medical device of the present invention overcomes the long-standing problem of typical echogenic coatings appearing similar to the shape and/or material of a bone or a titanium screw when viewed using ultrasonic imaging. In particular, a standard echogenic coating with no shape and/or pattern formed by echogenic material may be generally coated over a wide or uniform surface of a catheter or needle, resulting in a generally elongated and round or cylindrical echogenic coating that appears similar to the shape and/or material of a bone or a titanium screw when viewed using ultrasonic imaging. By providing at least one shape and/or pattern in the echogenic coating, the present inventors have found that the echogenically enhanced medical device of the present invention can be definitively identified when viewed using ultrasonic imaging in order to guide and confirm the position of the medical device within the patient's body.

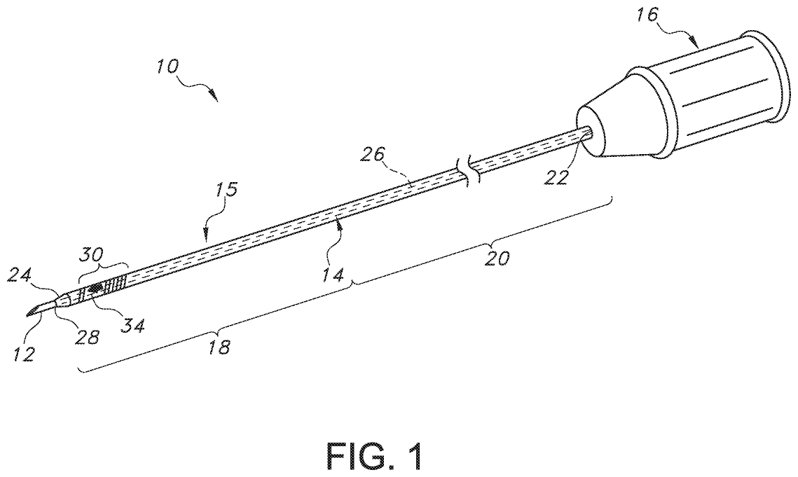

The specific features of the echogenically enhanced medical device of the present invention may be better understood with reference to FIGS. 1-5.

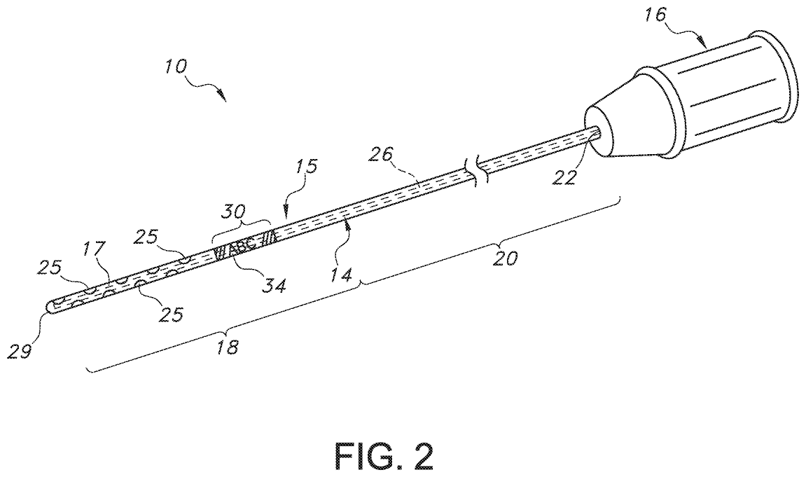

Referring now to the drawings, FIGS. 1 and 2 illustrate various embodiments of an echogenic catheter assembly 10 according to the present disclosure. For example, as shown, the catheter assembly 10 includes a catheter 14 having a proximal end 22 and a distal end 24. More specifically, as shown in FIG. 1, the catheter assembly 10 may be an over-the-needle (OTN) catheter assembly, i.e. the catheter 14 is coaxially mounted to the needle 12. Thus, the catheter assembly 10 may be configured such that the catheter 14 and needle 12 can be simultaneously inserted into a patient. Alternatively, as shown in FIG. 2, the catheter 14 may be used without a needle 12. In addition, the catheter 14 (and/or the needle 12) defines a lumen 26 extending from the proximal end 22 to the distal end 24 of the catheter 14. Thus, the catheter 14 is configured to deliver a treatment fluid to a targeted site, e.g. a nerve bundle, within the patient via the lumen 26. More specifically, as shown in FIG. 1, the catheter assembly 10 may include an open distal tip 28 such that the needle 12 may extend beyond the open distal tip 28.

In alternative embodiments, as shown in FIG. 2, the catheter assembly 10 may include a closed distal tip 29, e.g. depending on the desired delivery application of the treatment fluid to the patient. More specifically, as shown, the catheter assembly 10 may include a closed distal tip 29 without the needle 12. In such an embodiment, the catheter 14 may contain one or more infusion holes 25 configured on an outer surface 17 thereof so as to deliver a treatment fluid to a targeted site within a patient via the lumen 26 of the catheter 14. In addition, it should also be understood that the catheter assemblies according to the present disclosure may optionally include one or more infusion holes 25 as well as the open distal tip 28 for administering a treatment fluid to a patient.

More specifically, in certain embodiments, the proximal end 22 of the catheter 14 may include a hub 16 configured thereon for mating communication with a fluid delivery device (not shown) such that a treatment fluid can be delivered to a targeted site within a patient via the lumen 26 and the open distal tip 28 and/or the infusion holes 25 of the catheter 14 and/or needle 12. The fluid delivery device as described herein may be any suitable device known in the art, such as a pump, reservoir, syringe, or the like. Further, the hub 16 may have any conventional configuration, such as a Luer-lock fitting.

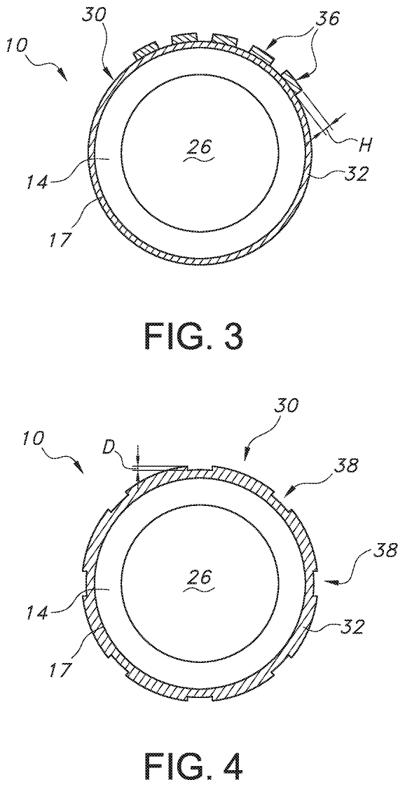

Referring still to FIGS. 1-2, a body portion 15 of the echogenic catheter assembly 10 that is configured to be inserted within human and/or animal tissue includes at least one structured echogenic portion 30 configured to enhance ultrasonic imaging of the catheter assembly 10. More specifically, as shown in FIGS. 3-4, the structured echogenic portion 30 may be formed as a structured coating 32 of an echogenic material on a portion of the outer surface 17 of the catheter 14 or on a portion of the needle 12. Additionally or alternatively, the structured echogenic portion 30 may be integrally formed as a portion of the catheter 14 itself, i.e., at least a portion 30 of the catheter 14 is formed from an echogenic material. The echogenic portion 30 can further include at least one structured shape or discontinuous pattern 34 formed in or on the echogenic portion 30. The at least one structured shape or discontinuous pattern 34 of the echogenic portion 30 is configured to be identifiable such that the insertion path and/or position of the echogenic catheter assembly 10 within the patient's body can be easily identified by ultrasonic imaging. For instance, as shown in FIGS. 1-2 and 5, the structured echogenic portion 30 can be located at or adjacent to the distal end or tip 24 of the catheter 14 or the distal end of the needle 12.

The at least one structured echogenic portion 30 of the echogenically enhanced devices of the present invention have useful ultrasonic scattering properties. When sonically imaged, e.g., using ultrasound imaging, the echogenic portion 30 of the echogenically enhanced device, e.g., catheter assembly 10, creates a high contrast with the medium in which it is inserted. The echogenic material may include micro-solids, e.g., microspheres, suspended in a coating solution that is applied to medical device to form the echogenic portion 30. In some embodiments, additionally or alternatively to suspended micro-solids, contrast may be enhanced by entrapped gas or fluid in porous portions of the surface of the echogenic portion. The amount of contrast expected between different materials when viewed with ultrasonic imaging can be estimated by comparing the acoustic impedance of each of the materials. The acoustic impedance is defined as the product of the density of a material times the speed of sound in the material. The acoustic impedance (in units of 10⁶ kg·m⁻²·s⁻¹) of some common materials are listed as follows: Air-0.0004; water-1.48; muscle-1.7; bone-7.8; metal-75. For imaging a device in a medium, the level of observed contrast may be related to the ratio between the high acoustic impedance material (e.g., metal or bone) to the low acoustic impedance material (e.g., air). By examining the acoustic impedances listed above, one can determine that for an aqueous medium such as blood, air has a higher acoustic impedance ratio (1.48/0.0004=3700) and would be expected to provide higher contrast with the medium than would metal (75/1.48=51). Thus, providing the echogenic material on at least a portion of the medical device, e.g., the needle 12 of the catheter assembly 10, results in the echogenic portion 30 exhibiting a significantly higher contrast in an aqueous medium than would the metal material of the needle 12 itself.

The echogenic material that forms the echogenic portion of the present invention may be a solution or coating of polymeric material having suspended micro-solids, e.g., microspheres, that enhance the acoustic contrast compared to body tissue, and/or porous polymeric material, such as a microporous polymeric material. However, the present invention contemplates that any other suitable material having echogenic properties or effect in contrast to human body tissue may be used. The solution having suspended micro-solids, e.g., microspheres can contain materials that allow for improved adhesion to the medical device, workability of the echogenic coating, and/or enhance the processing of the echogenic coating. The micro-solids or microspheres may be a non-metallic material. The coating solution can be viscous or non-viscous. A porous polymeric echogenic material is configured to have hollow cavities, whether at the surface or interior of the material, which entrap gas and/or fluid in the porous portions of the surface of the porous polymeric material when inserted into a medium, e.g., an aqueous medium such as blood, or human and/or animal tissue. In some aspects of the present invention, the conditions at which the porous polymeric material is formed and/or treated for preparation of the echogenic material for the echogenic portion 30 can be varied in order to produce a desired pore size and/or pore volume. The pore size may be adjusted by any suitable method known in the art. In some aspects, the pore size is at least about 10 nanometers, such as at least about 50 nanometers, for example at least about 1 micrometer. In some aspects, the pore size is less than or equal to about 500 micrometers, such as less than or equal to about 100 micrometers, for example less than or equal to about 10 micrometers. The total pore volume and the pore size may be adjusted to entrap sufficient echogenic contrast agent as is commonly used in the art of ultrasonic imaging.

It should be understood that the echogenic material of the present invention is biocompatible. A biocompatible material does not generally cause significant adverse reactions (e.g., toxic or antigenic responses) in the body, whether it degrades within the body, remains for extended periods of time, or is excreted whole. Ideally, a biocompatible material will not induce undesirable reactions in the body as a result of contact with bodily fluids or tissue, such as allergic reaction, foreign body reaction (rejection), inflammatory reaction, blood clotting, tissue death, or tumor formation, for example.

A coating 32 of echogenic material can be applied to the echogenic catheter assembly 10 by any coating methods known in the art. Either batch or continuous coating methods may be used to apply echogenic coatings of the present invention. Suitable coating methods include, but are not limited to, dip-coating; spray-coating, immersion coating, and combinations thereof. In one aspect of the present invention, a single-step dip-coating process is used. After the coating is applied, it may be dried by methods known in the art. Suitable drying methods include, but are not limited to, hot air (i.e., application of heat), conduction drying, convection drying, ultraviolet irradiation, infrared irradiation, and microwave irradiation. Ideally, the coating and drying methods are selected to provide a substantially uniform coating, i.e., a coating layer with a substantially uniform thickness and low surface roughness, prior to additive or subtractive formation of the at least one shape or discontinuous pattern 34 of the echogenic portion 30 as described in further detail below.

In some aspects, the dry thickness of the echogenic coating 32 prior to additive or subtractive formation of the at least one shape or discontinuous pattern 34 is at least about 1 micrometer, such as at least about 10 micrometers. The dry thickness of the echogenic coating 32 may be selected based on the size of the medical device, e.g., catheter 14, onto which the echogenic coating 32 is provided. For example, a 24-gauge catheter having an outer diameter of about 1 mm may not feasibly have an echogenic coating with a dry thickness of about 1 mm, because such a coating would increase the outer diameter of the echogenic portion to about 3 mm, three times as large as the initial outer diameter of the 24-gauge catheter, which may render it unsuitable for its intended purpose. Similarly, the thickness of the echogenic coating 32 may be selected based on the size of an introducer through which the medical device may need to be inserted, or other size considerations when the medical device must be used in conjunction with another apparatus.

Additionally or alternatively, as described above, the structured echogenic portion 30 can be a portion of the device, e.g., catheter 14, that is formed from an echogenic material. At least a portion of the device, e.g., catheter 14, may be prepared by shaping an echogenic material as desired to form at least a portion of the body portion of the device (e.g., tube of catheter 14). Suitable shaping methods are known in the art and include, for example, extrusion, compression molding, and extrusion molding. Structural components that are formed from echogenic materials may offer advantages over non-echogenic structural components that are coated with echogenic coatings to enhance echogenicity, e.g., by providing higher levels of echogenicity and elimination of potential problems from the coating layer such as cracking and delamination of the coating.

The structured echogenic portion 30 includes at least one structured shape or discontinuous pattern 34 in the echogenic portion 30 formed through additive methods, subtractive methods, or a combination thereof. For example, the structured shape or discontinuous pattern 34 can be formed by additive methods such as by printing echogenic material onto the device, e.g., needle 12 or catheter 14. The additive printing can be performed by inkjet printing, aerosol jet printing, direct inkjet printing, pressurized needle printing, volumetric pressure ink application and/or any other suitable printing process or additive manufacturing process capable of printing with an echogenic material. In some aspects of the invention, the additive manufacturing process may include, for example, directed energy deposition, direct laser deposition, or any other suitable additive manufacturing process. By using additive manufacturing, the various components of the structured shape or pattern 34 can be printed onto the catheter 14 or needle 12 in thin layers so as not to disturb the overall efficacy of the catheter 14 or needle 12, e.g., so as not to disturb the overall efficacy of the needle 12 in puncturing the necessary tissue of the patient. For example, in one embodiment, the at least one structured shape or pattern 34 may have a predetermined thickness or height H ranging from about 0.01 millimeters (mm) to about 0.5 mm. The additive printing of echogenic material can be performed on a catheter 14 or needle 12 as small as 24-gauge (i.e., having an outer diameter of about 0.040 inches or about 1 mm). Moreover, the additive manufacturing or printing of the additive structures 36 onto the catheter 14 or needle 12 is applied in specific shapes and at specific locations on the catheter 14 or needle 12. For instance, the additive manufacturing or printing of the additive structures 36 onto the catheter 14 or needle 12 can be placed at a particular distance from the distal tip of the catheter 14 or the needle 12, e.g., as near as about 0.1 mm from the distal tip. Additionally, the additive manufacturing or printing of the additive structures 36 onto the catheter 14 or needle 12 can be placed at a particular angular or radial position about the surface 17 of the catheter 14 or the needle 12. For example, as shown in FIG. 3, a plurality of additive structures 36 are radially disposed about the circumference of the catheter 14.

As shown in FIG. 3, the structured echogenic portion 30 can include additive structures 36 that are printed on the echogenic catheter device 10. The additive structures 36 can be additively printed onto an echogenic coating 32, as shown in FIG. 3, or can be directly printed onto the outer surface 17 of the catheter 14. The additive structures 36 are additively adhered onto the echogenic catheter device 10 to build up and form the shape and/or discontinuous pattern 34.

Additionally or alternatively, the at least one structured shape or discontinuous pattern 34 in the structured echogenic portion 30 can be formed through subtractive methods. For example, the echogenic catheter assembly 10 can be coated with an echogenic coating 32 over at least a portion of the catheter 14 as described above, and then the at least one structured shape or discontinuous pattern 34 can be removed from the coating 32 in a negative version of the shape and/or pattern. For example, at least one laser can be used to remove echogenic material from the echogenic coating 32 in a precise manner in the negative version of the structured shape and/or pattern. Other subtractive methods include, but are not limited to, laser ablation, plasma, chemical etching, and mechanical removal. For example, in one embodiment, the at least one structured shape or pattern 34 may have a predetermined depth D in the coating 32 as small as about 0.01 millimeters (mm). The subtractive formation of the shape and/or pattern into the coating 32 of echogenic material can be performed on a catheter 14 or needle 12 as small as 24-gauge (i.e., having an outer diameter of about 0.040 inches or about 1 mm). Moreover, the subtractive formation or printing of the subtractive areas 38 onto the echogenic coating 32 of the catheter 14 or needle 12 is formed in specific shapes and at specific locations on the catheter 14 or needle 12. For instance, the subtractive formation of the subtractive areas 38 onto the coating 32 on the catheter 14 or needle 12 can be placed at a particular distance from the distal tip of the catheter 14 or the needle 12, e.g., as near as about 0.1 mm from the distal tip. Additionally, the subtractive formation of the subtractive areas 38 onto the coating 32 of the catheter 14 or needle 12 can be placed at a particular angular or radial position about the surface 17 of the catheter 14 or the needle 12. For example, as shown in FIG. 3, a plurality of subtractive areas 38 are radially disposed about the circumference of the catheter 14.

As shown in FIG. 4, the structured echogenic portion 30 can be formed by an echogenic coating 32 surrounding the outer surface 17 of the catheter 14, where the echogenic coating 32 includes subtracted areas 38 that have been removed from the coating 32 to form a structured shape or pattern 34. In other aspects of the invention (not shown), a structured echogenic portion 30 can be formed by an echogenic coating 32 having subtracted areas 38 removed from the coating 32 and further having additive structures 36 printed or otherwise additively formed onto or around the echogenic coating 32.

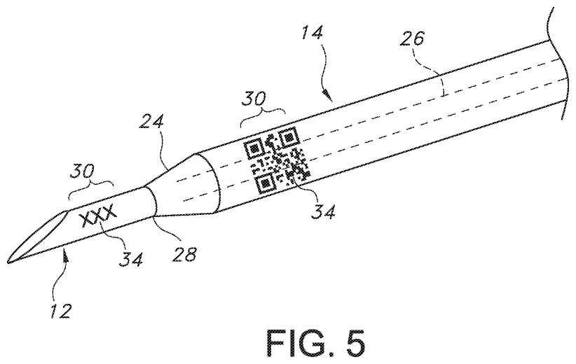

As shown in FIGS. 1-2 and 5, the at least one shape or discontinuous pattern 34 in the echogenic portion 30 can be in the form of one or more shapes, e.g., polygonal or other geometric shapes, arrow, bullseye, check mark, or other easily identifiable shape; alphanumeric characters, e.g., numbers, words, random or coded mixtures of numbers and/or letters, such as a serial number, date, product code, location code, expiration date, etc.; QR code(s); bar code(s); patterns, e.g., stripes, dash-dot sequences, swirled or curved lines/shapes, or any other repeating or non-repeating sequence of shapes; or any other suitable character or image, such as a company logo or name, that is suitable for positively identifying the device when viewed by ultrasonic imaging. For instance, FIG. 1 illustrates an arrow shape 34. FIG. 2 illustrates the letters “A B C” in the echogenic portion 30 of the catheter. FIG. 5 illustrates the letters/symbols “XXX” in the echogenic portion 30 of the needle 12 and a QR code in the echogenic portion 30 of the catheter 14.

By including at least one shape and/or discontinuous pattern 34 in the echogenic portion 30 of the echogenic catheter assembly 10, the present invention overcomes the long-standing problem of typical echogenic coatings appearing similar to the shape and/or material of a bone or a titanium screw when viewed using ultrasonic imaging. In particular, a standard echogenic coating with no shape and/or pattern formed by echogenic material may be generally coated over a wide surface of a catheter or needle, resulting in a generally elongated and round or cylindrical echogenic coating that appears similar to the shape and/or material of a bone or a titanium screw when viewed using ultrasonic imaging.

Although an echogenic catheter assembly 10 is illustrated in FIGS. 1-5, it is to be understood that the present invention contemplates an echogenic portion 30 having at least one shape or discontinuous pattern 34 as described above on any medical device assembly that is configured to be viewed using ultrasonic imaging. For example, a medical device or medical device assembly having an echogenic portion as described by the present invention can include, but is not limited to, a catheter; a needle; a cannula or introducer; a guidewire; an electrical lead; an implantable device; a probe, such as a radiofrequency ablation probe or any other insertable probe; an implantable fluid delivery port; and a breathing tube. The body portion 15 of the medical device in or on which the echogenic portion is formed may be from one or more of metal material, plastic material, ceramic material, and resin-based material.

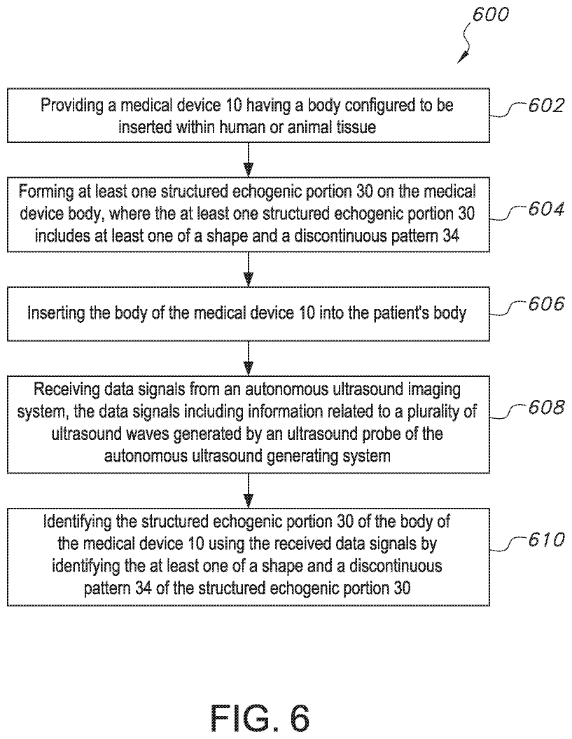

FIG. 6 shows a method 600 for preparing an echogenically enhanced medical device 10 and identifying the echogenically enhanced medical device 10 within a patient's body. First, in step 602, a medical device 10, e.g., a catheter assembly, is provided. The medical device 10 has a body portion 15 configured to be inserted within human or animal tissue. The body portion 15 of the medical device 10 has a first acoustic impedance. Then, in step 604, at least one structured echogenic portion 30 having a second acoustic impedance is formed on the medical device body portion 15. The structured echogenic portion 30 is configured to enhance ultrasonic imaging of the medical device body portion 15 when the body portion 15 is inserted into a patient. The at least one structured echogenic portion 30 formed in step 604 includes at least one of a shape and a discontinuous pattern 34. In step 606, the body portion 15 of the medical device 10, including the echogenically enhanced portion 30, is inserted into the patient's body. Then, in step 608, the method includes a step of receiving data signals from an autonomous ultrasound imaging system (not shown). The data signals include information related to a plurality of ultrasound waves generated by an ultrasound probe of the autonomous ultrasound generating system. For instance, data signals include information related to a plurality of ultrasound waves that are reflected off the echogenically enhanced portion 30 of the medical device body portion 15 such as the at least one shape and/or discontinuous pattern 34. Then, step 610 includes identifying the structured echogenic portion 30 of the body portion 15 of the medical device 10 using the received data signals by identifying the at least one of a shape and a discontinuous pattern 34 of the structured echogenic portion 30.

This written description uses examples to disclose the invention, including the best mode, and also to enable any person skilled in the art to practice the invention, including making and using any devices or systems and performing any incorporated methods. The patentable scope of the invention is defined by the claims and may include other examples that occur to those skilled in the art. Such other examples are intended to be within the scope of the claims if they include structural elements that do not differ from the literal language of the claims or if they include equivalent structural elements with insubstantial differences from the literal language of the claims.

What is claimed is:

1. An echogenically enhanced medical device comprising:
a body portion configured to be inserted within human or animal tissue, the body portion having a first acoustic impedance;
wherein the body portion further comprises at least one structured echogenic portion having a second acoustic impedance configured to enhance ultrasonic imaging of the medical device when inserted into a patient,
wherein the at least one structured echogenic portion comprises at least one of a structured shape and a discontinuous pattern.

2. The echogenically enhanced medical device of claim 1, wherein the body portion comprises one of: a needle; a catheter; a cannula; a guidewire; an electrical lead; an implantable device; a probe; an implantable fluid delivery port; a breathing tube.

3. The echogenically enhanced medical device of claim 1, wherein the body portion is formed from one or more of metal material, plastic material, ceramic material, and resin-based material.

4. The echogenically enhanced medical device of claim 1, wherein the second acoustic impedance is different from the first acoustic impedance.

5. The echogenically enhanced medical device of claim 1, wherein the at least one echogenic portion is formed from a material that is different from a material of the body portion.

6. The echogenically enhanced medical device of claim 1, wherein the discontinuous pattern of the echogenic portion is formed by additive or subtractive methods.

7. The echogenically enhanced medical device of claim 6, wherein the structured shape or discontinuous pattern is formed by printing an echogenic material onto the body portion.

8. The echogenically enhanced medical device of claim 6, wherein the structured shape or discontinuous pattern is imprinted by using a laser to remove material in the echogenic portion.

9. The echogenically enhanced medical device of claim 1, wherein the at least one structured shape or discontinuous pattern is a symbol in the form of an arrow, an alphabetical letter, a number, a serial number, a date, a bullseye, a QR code, a bar code, a logo, or other shape configured to indicate the identity of the medical device.

10. The echogenically enhanced medical device of claim 1, wherein the at least one structured shape or discontinuous pattern is a combination of alphabetical letters and/or numbers forming a date.

11. The echogenically enhanced medical device of claim 1, wherein the structured echogenic portion is formed by coating a portion of the body portion with an echogenic material.

12. The echogenically enhanced medical device of claim 1, wherein the echogenic portion is a portion of the body portion formed from an echogenic material.

13. A method for preparing an echogenically enhanced medical device comprising the steps of:
providing a medical device having a body portion configured to be inserted within human or animal tissue, the body portion having a first acoustic impedance;
forming a structured echogenic portion having a second acoustic impedance, wherein the structured echogenic portion is configured to enhance ultrasonic imaging of the medical device when inserted into a patient,
wherein the at least one structured echogenic portion comprises at least one of a shape and a discontinuous pattern.

14. The method of claim 13, wherein the step of forming a structured echogenic portion includes applying a coating of an echogenic material onto at least a portion of the body portion of the medical device.

15. The method of claim 14, further comprising a step of subtractively removing echogenic material from the echogenic coating to form the at least one of a shape and a discontinuous pattern.

16. The method of claim 14, further comprising a step of printing at least one additive structure onto the coating of echogenic material to form at least one of a shape and a discontinuous pattern.

17. The method of claim 13, wherein the at least one structured shape or discontinuous pattern is a symbol in the form of an arrow, an alphabetical letter, a number, a serial number, a date, a bullseye, a QR code, a barcode, a logo, or other shape configured to indicate the identity of the medical device.

18. The method of claim 13, wherein the at least one structured shape or discontinuous pattern is a combination of alphabetical letters and/or numbers forming a date.

19. The method of claim 13, wherein the at least one of a shape and a discontinuous pattern is additively formed on the body portion of the medical device.

20. The method of claim 19, wherein at least one additive structure is printed on the body portion of the medical device to form the at least one of a shape and a discontinuous pattern.

21. A method for identifying an echogenically enhanced medical device within a patient's body, the method comprising the steps of:
providing a medical device having a body portion configured to be inserted within human or animal tissue, the body portion having a first acoustic impedance;
forming at least one structured echogenic portion on the medical device body portion, the structured echogenic portion having a second acoustic impedance, wherein the structured echogenic portion is configured to enhance ultrasonic imaging of the medical device when inserted into a patient, wherein the at least one structured echogenic portion comprises at least one of a shape and a discontinuous pattern;
inserting the body portion of the medical device into the patient's body;
receiving data signals from an autonomous ultrasound imaging system, the data signals comprising information related to a plurality of ultrasound waves generated by an ultrasound probe of the autonomous ultrasound generating system; and
identifying the structured echogenic portion of the body portion of the medical device using the received data signals by identifying the at least one of a shape and a discontinuous pattern of the structured echogenic portion.
